(19) United States
(12) Patent Application Publication
(10) Pub. No.: US 2024/0170147 A1
(43) Pub. Date: May 23, 2024

(54) MACHINE LEARNING MODELS FOR PREDICTION OF UNPLANNED CESAREAN DELIVERY

(71) Applicant: Tel HaShomer Medical Research Infrastructure and Services Ltd., Ramat-Gan (IL)

(72) Inventors: Abraham TSUR, Ramat-Gan (IL); Raanan MEYER, Tel Aviv (IL); Roni EILENBERG, Kfar Saba (IL)

(73) Assignee: Tel HaShomer Medical Research Infrastructure and Services Ltd., Ramat-Gan (IL)

(21) Appl. No.: 18/280,254

(22) PCT Filed: Mar. 3, 2022
(86) PCT No.: PCT/IL2022/050244

§ 371 Date: Sep. 4, 2023

Related U.S. Application Data
(60) Provisional application No. 63/156,872, filed on Mar. 4, 2021.

(51) Int. Cl. G16H 50/20; A61B 5/00; A61B 8/08; G16H 50/30

(52) U.S. Cl. CPC G16H 50/20; A61B 5/435; A61B 8/0866; G16H 50/30

(57) ABSTRACT

There is provided a computer implemented method of training a machine learning model for prediction of unplanned Cesarean Delivery (uCD), comprising: creating or receiving a multi-record training dataset, wherein a record comprises: at least one fetal biometric parameter of a sample fetus obtained by an ultrasonography device, at least one personal parameter of a sample mother of the sample fetus, and a ground truth indicating whether a birth of the sample fetus by the sample mother was a uCD, and training the machine learning model on the multi-record training dataset for generating an outcome indicating likelihood of uCD for a target mother in response to an input of at least one fetal biometric parameter of a target fetus of the target mother and at least one personal parameter of the target mother.

### RELATED APPLICATION

This application claims the benefit of priority of U.S. Provisional Patent Application No. 63/156,872 filed on Mar. 4, 2021, the contents of which are incorporated herein by reference in their entirety.

### BACKGROUND

The present invention, in some embodiments thereof, relates to machine learning models and, more specifically, but not exclusively, to machine learning models for prediction of unplanned cesarean delivery.

Unplanned cesarean delivery (uCD) during attempted vaginal delivery is associated with higher morbidity and mortality rates compared with elective CD.

### SUMMARY

According to a first aspect, a computer implemented method of training a machine learning model for prediction of unplanned Cesarean Delivery (uCD), comprises: creating or receiving a multi-record training dataset, wherein a record comprises: at least one fetal biometric parameter of a sample fetus obtained by an ultrasonography device, at least one personal parameter of a sample mother of the sample fetus, and a ground truth indicating whether a birth of the sample fetus by the sample mother was a uCD, and training the machine learning model on the multi-record training dataset for generating an outcome indicating likelihood of uCD for a target mother in response to an input of at least one fetal biometric parameter of a target fetus of the target mother and at least one personal parameter of the target mother.

According to a second aspect, a computer implemented method of prediction of uCD, comprises: feeding at least one fetal biometric parameter of a target fetus of a target mother obtained by an ultrasonography device and at least one personal parameter of the target mother into a machine learning model trained according to the first aspect, and obtaining an indicating likelihood of uCD for a target mother as an outcome of the machine learning model.

According to a third aspect, a system for prediction of uCD, comprises: at least one processor executing a code for: feeding at least one fetal biometric parameter of a target fetus of a target mother obtained by an ultrasonography device and at least one personal parameter of the target mother into a machine learning model, and obtaining an indicating likelihood of uCD for a target mother as an outcome of the machine learning model, wherein the machine learning model is trained on a multi-record training dataset, wherein a record comprises: at least one fetal biometric parameter of a sample fetus of a sample mother obtained by an ultrasonography device, at least one personal parameter of the sample mother, and a ground truth indicating whether a birth of the sample fetus by the sample mother was a uCD.

In a further implementation form of the first, second, and third aspects, the at least one fetal biometric parameter and the at least one personal parameter are for the fetus and/or sample mother at time of admission of the mother to labor.

In a further implementation form of the first, second, and third aspects, the at least one fetal biometric parameter of the sample fetus obtained by the ultrasonography device depicts a historical gestational age of the sample fetus, and further comprising adapting the at least one fetal biometric parameter to an adapted at least one fetal biometric parameter depicting a current gestational age at time of admission to labor, wherein the record includes the adapted at least one fetal biometric parameter.

In a further implementation form of the first, second, and third aspects, the at least one personal parameter comprises at least one risk modifier.

In a further implementation form of the first, second, and third aspects, the at least one personal parameter is based on a state of a cervix of the mother.

In a further implementation form of the first, second, and third aspects, the at least one personal parameter is represented as a continuous value.

In a further implementation form of the first, second, and third aspects, the at least one fetal biometric parameter is selected from a group comprising: estimated fetal weight, head circumference, and biparietal diameter.

In a further implementation form of the first, second, and third aspects, the at least one personal parameter is selected from a group comprising: number of prior vaginal deliveries, cervical dilation, spontaneous onset of labor, cervical effacement, maternal BMI at admission to labor, cervical ripening required, gestational age at admission, maternal height, fetal head station, and maternal age.

In a further implementation form of the first, second, and third aspects, the ground truth is a label indicating that the birth of the sample feature is uCD or vaginal delivery.

In a further implementation form of the first, second, and third aspects, the machine learning model comprises a binary classifier that generates the outcome indicative of uCD or vaginal delivery.

In a further implementation form of the first, second, and third aspects, the outcome indicating likelihood of uCD generated by the machine learning model comprises a predicted probability of uCD.

In a further implementation form of the first, second, and third aspects, further comprising excluding records from the training dataset associated with personal parameters that include values of at least one of: a delivery of more than one fetus, non-vertex, no trial of vaginal delivery, delivery at <34 weeks of gestation, terminal of pregnancy, fetal demise, and prior cesarean delivery.

In a further implementation form of the first, second, and third aspects, further comprising including records in the training dataset with personal parameters including values of: a singleton pregnancy, >=34 weeks of gestation, admitted for vaginal delivery, and fetus at vertex presentation.

In a further implementation form of the first, second, and third aspects, further comprising computing relative importance of the at least one fetal biometric parameter and/or for the at least one personal parameter in generating the outcome by the ML model, selecting a subset of the at least one fetal biometric parameter and/or for the at least one personal parameter having a relative importance above a threshold, wherein records of the selected subset are included in the multi-record training dataset used to train the ML model.

In a further implementation form of the first, second, and third aspects, further comprising, in response to obtaining the outcome of the machine learning model indicating high likelihood of uCD, treating the target mother by performing a cesarean delivery surgical procedure.

In a further implementation form of the first, second, and third aspects, further comprising, in response to obtaining the outcome of the machine learning model indicating low likelihood of uCD, treating the target mother by performing a vaginal delivery.

In a further implementation form of the first, second, and third aspects, further comprising: when the at least one fetal biometric parameter of the target fetus obtained by the ultrasonography device is of a historical gestational age of the target fetus, adapting the at least one fetal biometric parameter to an adapted at least one fetal biometric parameter depicting a current gestational age at time of admission to labor, wherein feeding comprises feeding the adapted at least one fetal biometric parameter into the machine learning model.

In a further implementation form of the first, second, and third aspects, the at least one fetal biometric parameter and the at least one personal parameter are for the fetus and/or sample mother at time of admission of the mother to labor.

In a further implementation form of the first, second, and third aspects, further comprising applying a machine learning model interpretability process for computing relative contribution of each one of the at least one fetal biometric parameter and the at least one personal parameter towards the outcome generated by the machine learning model, and presenting an indication of the relative contribution on a display.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a block diagram of components of a system for training a machine learning model for prediction of unplanned cesarean delivery and/or for inference by the machine learning model, in accordance with some embodiments of the present invention;

FIG. 2 is a flowchart of a method of training a machine learning model for prediction of unplanned cesarean delivery, in accordance with some embodiments of the present invention;

FIG. 3 is a flowchart of a method of inference by a machine learning model for prediction of unplanned cesarean delivery, in accordance with some embodiments of the present invention; and

FIGS. 4-18 relate to a study performed by Inventors for evaluation of a machine learning model for prediction of unplanned cesarean delivery, in accordance with some embodiments of the present invention.

### DETAILED DESCRIPTION

The present invention, in some embodiments thereof, relates to machine learning models and, more specifically, but not exclusively, to machine learning models for prediction of unplanned cesarean delivery.

An aspect of some embodiments of the present invention relates to system, methods, apparatus (i.e., a computing device), and/or code instructions (stored on a memory and executable by one or more hardware processors) for training a machine learning (ML) model for prediction of unplanned Cesarean Delivery (uCD) of a target subject. A multi-record training dataset is created and/or received and/or accessed. A record of the multi-record training dataset includes one or more fetal biometric parameters of a sample fetus obtained by an ultrasonography device. When the fetal biometric parameter(s) are obtained in advance (e.g., of the time of admission of the mother to labor), depicting a historical gestational age of the sample fetus, the fetal biometric parameter(s) may be adapted to the current gestational age of the sample fetus (e.g., at the time of admission of the mother). The record includes one or more personal parameters of a sample mother of the sample fetus, for example, risk modifiers and/or based on a state of a cervix of the mother (e.g., based on a cervix score, based on a Bishop score). The record includes a ground truth indicating whether a birth of the sample fetus by the sample mother was a uCD and/or whether the birth was non-uCD (e.g., vaginal delivery). The machine learning model is trained on the multi-record training dataset.

An aspect of some embodiments of the present invention relates to system, methods, apparatus (i.e., a computing device), and/or code instructions (stored on a memory and executable by one or more hardware processors) for computing a prediction of uCD in a target subject. A ML model trained on a multi-record training dataset (e.g., as described herein) is provided. One or more fetal biometric parameters of a target fetus of the target mother and one or more personal parameters of the target mother are fed into the ML model. When the fetal biometric parameter(s) are obtained in advance (e.g., of a time of admission of the mother), depicting a historical gestational age of the sample fetus, the fetal biometric parameter(s) may be adapted to a current gestational age of the sample fetus (e.g., at the time of admission of the mother). The adapted fetal biometric parameter(s) are fed into the ML model. An outcome indicating likelihood of uCD for the target mother is obtained from the ML model.

At least some implementations of the systems, methods, devices, and/or code instructions (stored on a data storage device and executable by processors and/or implemented in circuitry) described herein address the technical problem of predicting likelihood of unplanned cesarean delivery. Approximately 15% of vaginal delivery (VD) attempts in the Unites States eventually lead to an unplanned cesarean delivery (uCD) (1, 2). The rates of uCD are even higher among some subgroups such as nulliparous women and those requiring labor induction, reaching reported rates of 21% (3, 4) and 26% (5) respectively. The most common indications for uCDs are non-reassuring fetal status and arrest of dilatation or descent (1, 6). uCD during attempted VD is associated with higher morbidity and mortality rates compared with elective cesarean deliveries (CD) (7-9). These include intraoperative complications, maternal trauma, hemorrhage, febrile morbidity, intensive care unit admission, low neonatal Apgar scores, neonatal intensive care unit admission and other complications (10-12). Precise prelabor identification of women at high risk of uCDs could potentially contribute to reduction of maternal and neonatal morbidity related to uCD on the one hand, while providing reassurance to the majority of women that are at low risk for uCD.

At least some implementations of the systems, methods, devices, and/or code instructions described herein improve the technical field of medicine by predicting likelihood of unplanned cesarean delivery. At least some implementations of the systems, methods, devices, and/or code instructions described herein improve the technical field of machine learning, by providing features that are used to train a ML model, and/or fed during inference into the ML model, for predicting likelihood of unplanned cesarean delivery.

The features may be features that are available at admission to labor. The features may be for example, for cases of singleton deliveries ≥34 gestational weeks, or other gestational ages. As described herein, for example in the “Examples” section, Inventors performed a study and discovered that the features used to train the ML model provide excellent clinical discrimination by the ML model. The model predictive accuracy measured by the area under the curve (AUC) is 0.832. Clinical discrimination is demonstrated by a positive predictive value of 58% for unplanned cesarean delivery among women in the 100^{th} percentile risk group, and a negative predictive value of ≥99% in the <50^{th} percentile risk group. As described in the “Examples” section, all women carrying a singleton gestation at ≥34 weeks of gestation who were admitted for vaginal delivery with fetus at vertex presentation were included. Women with a prior cesarean or intrauterine fetal demise/termination of pregnancy were excluded. The model development cohort consisting of deliveries from March 2011 to May 2019 was divided to training (80%) and validation (20%) sets. In addition, a separate cohort of deliveries from June 2019 to June 2020 served as a test set. Maternal risk modifiers, fetal biometric parameters and Bishop scoring served as features for developing the model. Feature selection was performed using Random Forest machine learning algorithm. The study population included 67,121 women, of which 4,125 (6.1%) underwent uCD. The training, validation and test sets comprised 48,084, 12,016 and 7,021 cases, respectively. The final model consisted of thirteen features, based on prediction importance. The area under the receiver operator curve (AUC) for the training, validation and test sets were 0.874, 0.839 and 0.832, respectively. Clinically, the model showed a positive predictive value of 58% for uCD among women in the 100th percentile group, and a negative predictive value of ≥99% in the <50th percentile group. Among subgroups with higher pretest probability of uCD namely nulliparous, obese, advanced maternal age, and those requiring labor induction. The positive predictive value (PPV) of the 100th percentile group was even higher: 59%, 57%, 63% and 66%, while the negative predictive value (NPV) among women <50^{th} percentile remained reassuring: 98%, 98%, 100% and 98%. The ML approach provided comprehensive prediction that was stable across validation and test sets, with clinical discrimination preserved across gestational age ≥34-42 (e.g., from 34+0 weeks until 42+0) weeks of gestation, and clinical risk groups, for example, across groups with high pretest probability for uCD: nulliparous (3), obese (14), advanced maternal age (28), and those undergoing labor inductions (5). All features required for risk stratification by the model are available at time of admission to labor.

The machine learning model provides excellent clinical discrimination, identifying women at high risk for unplanned cesarean. The outcomes of the ML model may be used, for example, for providing reassurance to the majority of women and/or pregnancies, and/or for counseling women at time of admission to labor to inform their individualized decision making, weighing individual probabilities with individual preferences. The ML model may be trained for providing a comprehensive and stable prediction that is not limited to a specific subgroup or only term deliveries. The outcome of the ML model may be used, for example, for counseling women at time of admission to labor to inform their individualized decision making. A comprehensive prediction model of unplanned cesarean deliveries may contribute significantly to reduction of morbidity related both to emergent cesarean delivery and to potentially avoidable elective cesarean deliveries. The machine learning model described herein detects patterns derived from large, complex data, and provides a comprehensive prediction.

At least some implementations of the systems, methods, devices, and/or code instructions described herein improve upon existing approaches. Previous approaches focused on clinical tools for the prediction of uCD among specific subgroups of women at relatively high risk for uCD such as nulliparous (3), those undergoing labor induction (5, 13) or even more specifically obese women undergoing induction (14). Some of these studies also provided an online calculator (13, 14) or a nomogram (3, 5) that can be used by obstetricians or the women themselves. However, these approaches are valid only among specific subgroups, limiting their generalizability and wide clinical use. Furthermore, with the exception of the labor induction model by Rossi et al. (13), the referred models were limited to term deliveries (3, 5, 14). Other models used intrapartum assessment including ultrasound and fetal monitor, hence cannot serve for counseling at admission to labor (15-17). In contrast, at least some approaches described herein relate to an ML model designed for a wide range of different women, not necessarily having known high risk factors for uCD, for example, multiparous, those that are not undergoing labor induction, at a variety of gestational ages of the fetuses, and/or using ultrasound measurements made before admission to labor and/or other event indicating impending birth (e.g., by adjusting the ultrasound measurements as described herein).

The approach described herein based on the ML model improves over other prior approaches which have used other ML models. Machine learning approach are designed for detecting patterns derived from large, complex data (18, 19) and may provide a better and more comprehensive prediction in different settings, including obstetrics (20, 21). A recent study utilizing ML reported an AUC of 0.817 for uCD prediction (22). However, the developed model was based on neonatal biometry that is not available at time of admission to labor, and was limited to term deliveries and the authors did not provide a model for clinical use. In contrast, at least some approaches described herein relate to an ML model designed to use fetal biometric parameters that are available at time of admission to labor (or are computed as adapted fetal biometric parameters), not necessarily limited to term deliveries as other earlier gestational ages may be used, and/or is designed for clinical use.

At least some implementations described herein address technical challenges that have limited previous attempts from developing generalizable models. This enabled creating the ML model described herein may be used for a wide range of women (e.g., various gestational ages, various ages, and other variations described herein) carrying a singleton pregnancy with the possible exception of women attempting TOLAC (Trial Of Labor After Cesarean delivery).

One technical challenge is data availability. As described in the “Examples” section below, Inventors performed a study that used a meticulously labeled cohort including 99,579 deliveries with granular data captured across 10 years, which addressed the technical challenge of data availability.

Another technical challenge is the limited ability of logistic regression-based models to untangle complex interactions. As described above, previous approaches focused on developing models focusing on specific subgroups and/or specific GA, which reduced complexity of such interactions. These provides approaches limited generalizability and/or did not enable categorizing continuous features such as weight, BMI and/or maternal age. In contrast, the ML model described herein is designed for more general cases and/or for enabling using continuous features such as weight, BMI and/or maternal age. In the ML model described herein, features such as parity, labor induction, BMI and/or maternal age serve as predictive features interacting with other features contributing to comprehensive prediction of uCD, rather than serving only as inclusion or exclusion criteria as in prior approaches.

In another example, another earlier study for prediction of uCD had a cohort that was limited to term deliveries only, lacked a nomogram for clinical use, and was based on neonatal biometry not available at admission to labor, rather than the fetal ultrasound (22). In contrast, at least some embodiments described herein relate to an ML model trained on a cohort that included a wide range of gestational ages, included a nomogram for clinical use, and/or based on fetal biometric parameters which are available at admission to labor, and/or by computing adapted fetal biometric parameters computed for the time of admission to labor.

Improvements of embodiments described herein over prior approach include, for example, large, meticulously collected labor and/or delivery datasets, that include parameters based on sonographic assessment and/or birth outcomes used to train ML models. Another example of an improvement of the ML model described herein over exiting approaches is the stable performance across validation and/or test sets, and/or the stable clinical discrimination across gestational weeks and clinical subgroups. Yet another example of an improvement of the ML model described herein over exiting approaches is the availability of a comprehensive clinical tool than can serve for any singleton delivery from 34 weeks of gestational onwards, optionally with the exception of TOLAC.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

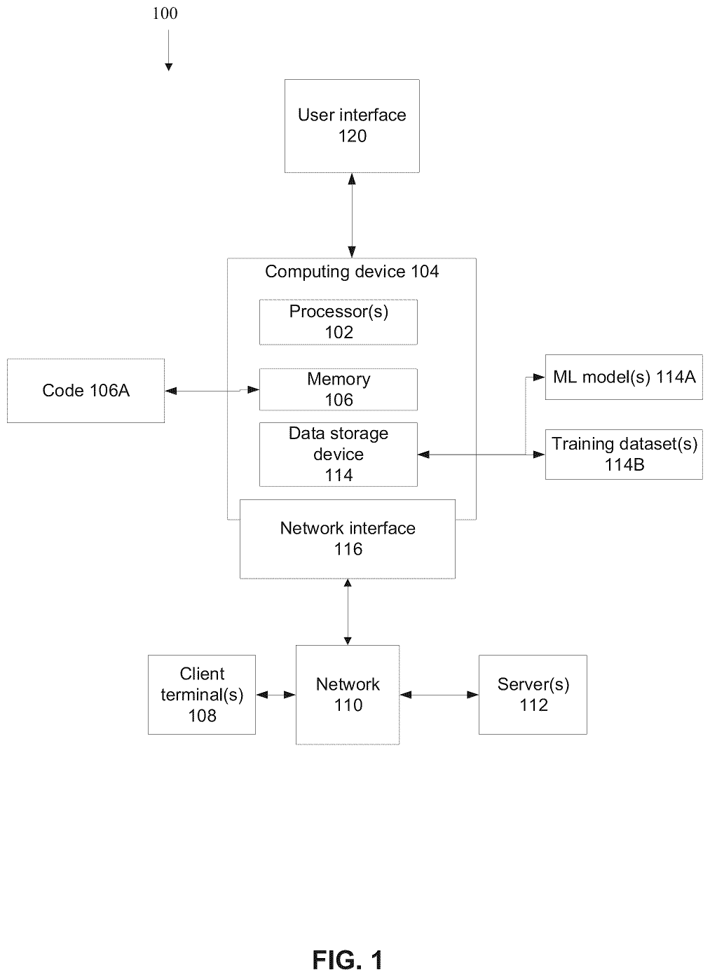

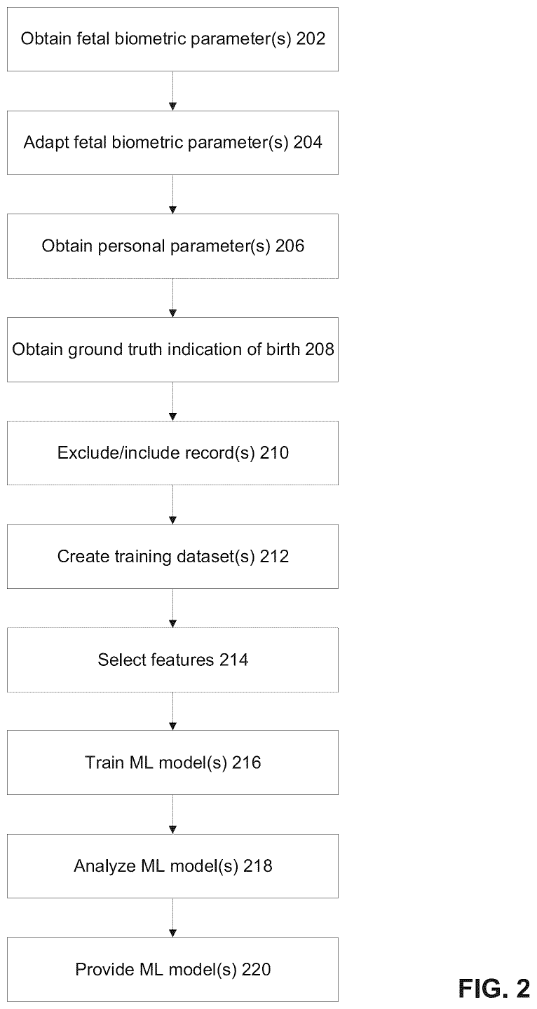

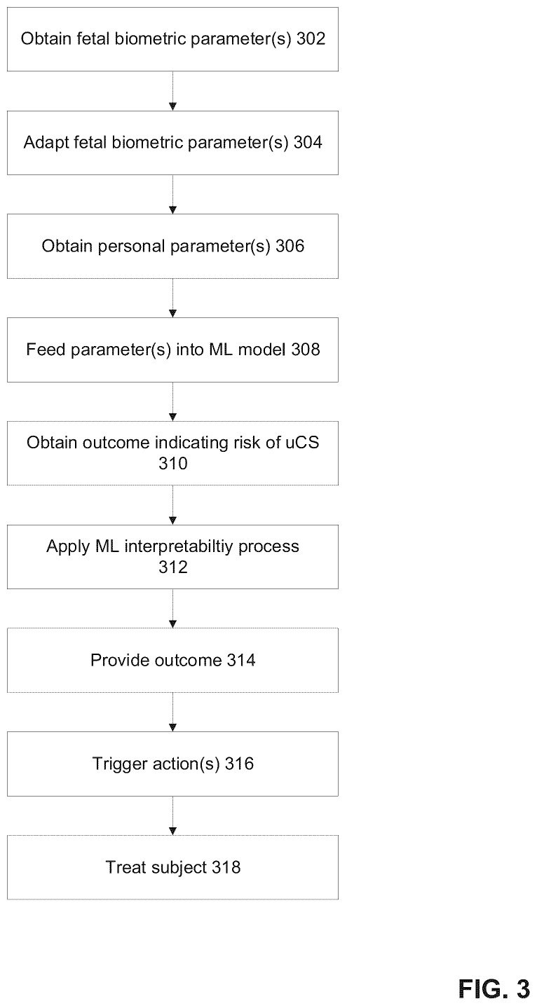

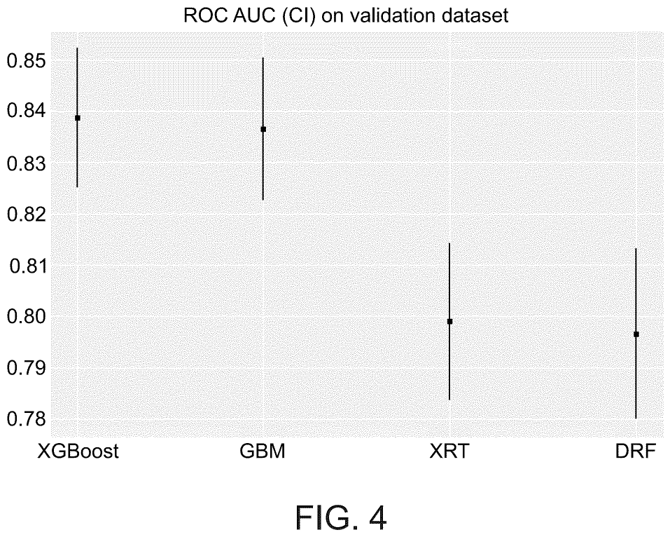

Reference is now made to FIG. 1, which is a block diagram of components of a system 100 for training a machine learning model for prediction of unplanned cesarean delivery and/or for inference by the machine learning model, in accordance with some embodiments of the present invention. Reference is also made to FIG. 2, which is a flowchart of a method of training a machine learning model for prediction of unplanned cesarean delivery, in accordance with some embodiments of the present invention. Reference is also made to FIG. 3, which is a flowchart of a method of inference by a machine learning model for prediction of unplanned cesarean delivery, in accordance with some embodiments of the present invention. Reference is also made to FIGS. 4-18, which relate to a study performed by Inventors for evaluation of a machine learning model for prediction of unplanned cesarean delivery, in accordance with some embodiments of the present invention.

System 100 may implement the acts of the method described with reference to FIGS. 2-18 by processor(s) 102 of a computing device 104 executing code instructions stored in a memory 106 (also referred to as a program store).

Computing device 104 may be implemented as, for example one or more and/or combination of: a group of connected devices, a client terminal, a server, a virtual server, a computing cloud, a virtual machine, a desktop computer, a thin client, a network node, and/or a mobile device (e.g., a Smartphone, a Tablet computer, a laptop computer, a wearable computer, glasses computer, and a watch computer).

Multiple architectures of system 100 based on computing device 104 may be implemented. For example:
A local architecture. Computing device 104 executing stored code instructions 106A may be implemented as a standalone device, for example, a client terminal, or a smartphone. Computing device 104 locally ML model 114A on training dataset 114B. The training may be personalized, for example, trained for a specific healthcare organization, and/or trained for a specific geographical area. It is noted that the ML model may be trained to handle different populations, rather than focusing on a specific population, such as women over a certain age, obese women, and the like. Different computing devices which may be used by different users may have different training datasets for training their own personalized ML models, for example, different healthcare organizations may create different training datasets using data from their respective organizations for generating personalized ML models to be used by each organization. Computing device 104 locally performs inference using ML model 114A using features available at time admission to provide an indication of unplanned cesarean delivery, for example, to help counsel mothers on making decisions, as described herein.
A centralized architecture. Computing device 104 executing stored code instructions 106A, may be implemented as one or more servers (e.g., network server, web server, a computing cloud, a virtual server) that provides centralized services (e.g., one or more of the acts described with reference to FIGS. 2-18) to one or more client terminals 108 over a network 110. For example, providing software as a service (SaaS) to the client terminal(s) 108, providing software services accessible using a software interface (e.g., application programming interface (API), software development kit (SDK)), providing an application for local download to the client terminal(s) 108, providing an add-on to a web browser running on client terminal(s) 108, and/or providing functions using a remote access session to the client terminals 108, such as through a web browser executed by client terminal 108 accessing a web site hosted by computing device 104. For example, computing device 104 centrally trains ML model 114A on training dataset 114B. Computing device 104 centrally performs inference using ML model 114A using features available at time admission to provide an indication of unplanned cesarean delivery, as described herein. This enables a centralized determination of unplanned cesarean delivery from different client terminals 108 used by different users in different locations, for example, to mothers using their smartphones at home to access an online calculator to determine their risk of uCD to help assess risk of an at-home birth versus hospital birth, and/or by healthcare staff of different hospitals to help counsel the mothers.
A combined local-central architecture. Computing device 104 may be implemented as a server that include locally stored code instructions 106A that implement one or more of the acts described with reference to FIGS. 2-18, while other acts described with reference to FIGS. 2-18 are handled by client terminal(s) 108 (e.g., external network connected devices). For example, ML model 114A is trained by computing device 104 using training dataset 114B, as described herein. ML model 114A is provided to each client terminal 108 for local inference, for example, for determining risk of uCD at the point of care, such as in the labor room, using a smartphone. Computing device 104 may train a single main ML model 114A which is provided to multiple different client terminals. Alternatively or additionally, computing device 104 may train a respective customized ML model 114A for each individual client terminal, or for each group of client terminals. Each respective customized trained ML is locally used by respective individual client terminals or respective groups.

Hardware processor(s) 102 of computing device 104 may be implemented, for example, as a central processing unit(s) (CPU), a graphics processing unit(s) (GPU), field programmable gate array(s) (FPGA), digital signal processor(s) (DSP), and application specific integrated circuit(s) (ASIC). Processor(s) 102 may include a single processor, or multiple processors (homogenous or heterogeneous) arranged for parallel processing, as clusters and/or as one or more multi core processing devices.

Memory 106 stores code instructions executable by hardware processor(s) 102, for example, a random access memory (RAM), read-only memory (ROM), and/or a storage device, for example, non-volatile memory, magnetic media, semiconductor memory devices, hard drive, removable storage, and optical media (e.g., DVD, CD-ROM). Memory 106 stores code 106A that implements one or more features and/or acts of the method described with reference to FIGS. 2-18 when executed by hardware processor(s) 102.

Computing device 104 may include a data storage device 114 for storing data, for example, ML model(s) 114A and/or training dataset(s) 114B, as described herein. Data storage device 114 may be implemented as, for example, a memory, a local hard-drive, virtual storage, a removable storage unit, an optical disk, a storage device, and/or as a remote server and/or computing cloud (e.g., accessed using a network connection).

Network 110 may be implemented as, for example, the internet, a local area network, a virtual network, a wireless network, a cellular network, a local bus, a point to point link (e.g., wired), and/or combinations of the aforementioned.

Computing device 104 may include a network interface 116 for connecting to network 110, for example, one or more of, a network interface card, a wireless interface to connect to a wireless network, a physical interface for connecting to a cable for network connectivity, a virtual interface implemented in software, network communication software providing higher layers of network connectivity, and/or other implementations.

Computing device 104 may communicate with client terminal(s) 108 and/or server(s) 112 over a network. Client terminal(s) 108 may be used, for example, by end users to provide features which are fed into the ML model 114A and/or for displaying the outcome of the ML model on a display of the client terminal. Server(s) 112 may, for example, provide features to computing device 104 for feeding into the ML model 114A, such as an electronic health record server (e.g., storing health records of the mothers), a picture archiving and communication system (PACS) server (e.g., storing ultrasound images and/or measurements of the fetus), and the like.

Computing device 104 includes and/or is in communication with one or more physical user interfaces 120 that include a mechanism for a user to enter data (e.g., enter features fed into the ML model) and/or view data (e.g., risk of uCD generated by the ML model). Exemplary user interfaces 120 include, for example, one or more of, a touchscreen, a display, a virtual reality display (e.g., headset), gesture activation devices, a keyboard, a mouse, and voice activated software using speakers and microphone.

Referring now back to FIG. 2, at 202, one or more fetal biometric parameter of a sample fetus of a sample mother are obtained by an ultrasonography device. Fetal biometric parameters are measurement of the sample fetus performed while the fetus is in the sample mother's womb, prior to birth (i.e., antenatal). The fetal biometric parameters are made using an ultrasonography device. The fetal biometric parameters may be done, for example, as part of medical examinations for assessing the state of the fetus, such as growth of the fetus, and/or to check for anatomical abnormalities.

Examples of fetal biometric parameters include: estimated fetal weight (EFW), head circumference, biparietal diameter, biparietal diameter (BPD), head circumference (HC), abdominal circumference (AC), femur length (FL), and amniotic fluid index (AFI).

Other measurements of the fetus made by ultrasound which may be obtained include sex of the fetus.

Fetal biometric parameters may be computed based on ultrasound measurements, for example, by code executed by a processor of an ultrasound workstation. For example, the estimated fetal weight (EFW) may be computed from other ultrasound measurements. The AFI may be calculated by summing the maximal vertical amniotic fluid pocket diameter in four quadrants of the uterus.

The fetal biometric parameter(s) may be obtained in near temporal proximity to the birth, optionally at time of admission of the mother to labor, and/or during other events that indicate impending birth, for example, contractions (e.g., excluding Braxton Hicks), rupture of the sac, fetal monitoring, and the like. The fetal biometric parameter(s) may be obtained, for example, no more than about 1 hour, or 4 hours, or 6 hours, or 12 hours, or 24 hours, or 36 hours, or 48 hours prior to the birth.

As used herein, the phrase “at time of admission to labor” refers to data obtained in near temporal proximity to the birth of the fetus, for example, no more than about 1 hour, or 4 hours, or 6 hours, or 12 hours, or 24 hours, or 36 hours, or 48 hours prior to the birth. The phrase “at time of admission to labor” may refer to other event(s) indicating impending birth, for example, onset of labor, contractions, rupture of sac and release of amniotic fluid, and the like.

The fetal biometric parameter(s) may be accessed, for example, from a health record of the sample subject, from a PACS server, from an external data storage device storing the parameters, from the ultrasound device (e.g., workstation, ultrasound machine), from a storage server, manually entered by a user, and the like.

The fetal biometric parameters may be represented using different scales, categories, and/or numerical systems, for example, on a continues scale, on a discrete scale, and/or as a category selected from multiple categories.

At 204, fetal biometric parameter(s) depicting a historical gestational age of the sample fetus may be adapted to depict a current gestational age of the fetus, for example, at the time of admission to labor and/or other event indicating impending birth. The adapted fetal biometric parameter(s) is included in the record.

For example, EFW may be adjusted to aEFW, BPD may be adjusted to aBPD, HC may be adjusted to aHC, AC may be adjusted to aAC, and/or FL may be adjusted to aFL.

The adaptation may be performed when the fetal biometric parameter(s) that are accessed are within a range and/or threshold from a time of admission to labor and/or other event indicating impending birth, for example, within a week, within 2 weeks, within 3 weeks, within 4 weeks, within 5 weeks, within 6 weeks, within 7 weeks, within 8 weeks, or other values.

The adaptation may be performed when the fetal biometric parameter(s) that are accessed are greater than a threshold from a time of admission to labor and/or other event indicating impending birth, for example, greater than a week, or 2 weeks, or a month, or 6 weeks, or 8 weeks, or greater or intermediate values.

The adaptation may be performed to obtain current values of the fetal biometric parameter(s) depicting the current gestational age of the fetus when current values are not available and/or cannot be obtained, and/or when historical values are available. Sonographic ultrasound evaluation of the fetus is not routinely performed at all centers worldwide at time of admission to labor. The adaptation is performed to obtain estimated fetal biometric parameters depicting the gestational age of the fetus at the time of admission to labor and/or other event indicating impending birth. The adaptation may be performed since fetal ultrasound biometry may change from the time of examination to the time of delivery.

An exemplary approach for adjusting sonographic biometry according to gestational age (GA) is now described. Additional details and/or exemplary approaches may be found, for example, with reference to (20). The approach may include: calculating the percentile of the respective fetal biometric parameter at the GA it was performed, for example, based on the Hadlock equations (26, 27), for example, by calculating the median expected value for the respective fetal biometric parameter for the GA on the day (or other proximal time) the ultrasound was performed. The respective fetal biometric parameter at admission to labor (and/or other event indicating impending birth) may be adjusted, for example, based on the normal distribution, and/or the percentile which was calculated in the previous step, and/or the median expected value for the respective fetal biometric parameter for the GA at the day of delivery (or other proximal time).

At 206, one or more personal parameters of the sample mother of the sample fetus are obtained. The personal parameters may be risk modifiers that modify risk of uCD. The personal parameters may be based on a state of a cervix of the mother, for example, based on a Bishop score. The Bishop score is a pre-labor scoring system to assist in predicting whether induction of labor will be required. The Bishop Score was developed by Professor Emeritus of Obstetrics and Gynecology, Dr. Edward Bishop, and was first published in August 1964. Other personal parameters associated with the sample mother may be used.

The term material parameter may be interchanged with the term personal parameter.

The personal parameters may be represented using different scales, categories, and/or numerical systems, for example, on a continues scale, on a discrete scale, and/or as a category selected from multiple categories.

Examples of personal parameter(s) include: number of prior vaginal deliveries, material weight, cervical dilation, mechanism of onset of labor (e.g., spontaneous, prelabor rupture, and induction), cervical effacement, maternal BMI at admission to labor, cervical ripening required, gestational age at admission, maternal height, fetal head station, and maternal age.

One or more of the above mentioned personal parameters may be represented as continuous values. For example, the material age may be represented as years and fraction of a year indicating number of days within the year. The material weight and/or maternal BMI may be represented, for example, as a whole number, to within one decimal of accuracy, to within two decimals of accuracy, and higher.

Examples of personal parameter(s) based on the state of the cervix of the mother include cervical dilation, cervical effacement, cervical consistency, cervical ripening required, cervical position, and fetal station.

The personal parameter(s) may be values indicating the state of the sample mother at time of admission to labor and/or other event indicating impending birth.

Examples of personal parameter(s) based on the Bishop score include: cervical dilation, cervical effacement, fetal station, and mode of start of delivery.

Examples of personal parameter(s) which may represent material risk modifiers include: age, prepregnancy weight, height, body mass index (BMI), smoking status and maternal comorbidities, obstetrical history, current pregnancy characteristics including gestational age (GA) at admission to labor, pregnancy complications (e.g., gestational diabetes mellitus, hypertensive disorders of pregnancy, intrahepatic cholestasis of pregnancy);

The BMI may be calculated as weight (kg)/height² (m). Weight may be reported by the parturient as pre-gestational and/or weight at admission to labor. Hypertensive disorders may be as defined according to the American College of Obstetricians and Gynecologists (ACOG) (23). Diabetic disorders may be as defined as pregestational diabetes, for example in accordance with the American Diabetes Association criteria (24), and/or gestational diabetes mellitus, for example, using the diagnostic thresholds established by Carpenter and Coustan (25). In cases that cervical ripening was required, either intracervical foley catheter and/or prostaglandin E2 may be used at the discretion of the treating physician.

The personal parameter(s) are obtained at time of admission of the sample mother to labor and/or other event indicating impending birth.

The personal parameter(s) may be obtained, for example, automatically from a health record of the sample subject and/or from another database, from sensor(s) that measure the subject (e.g., weight is obtained from a sensor associated with a scale), and/or manually entered by a user.

At 208, a ground truth indicating whether a birth of the sample fetus by the sample mother was a uCD, is generated.

The ground truth label may be, for example, a binary label indicating, for example, uCD and non-UCD, or uCD and vaginal delivery.

The ground truth label may be obtained, for example, automatically from a health record of the sample subject, and/or manually entered by a user.

At 210, records may be excluded from the training dataset and/or records may be included in the training dataset.

Optionally, records associated with one or more of the following are excluded from the training dataset: a delivery of more than one fetus, non-vertex presentation, no trial of vaginal delivery, delivery at <34 weeks of gestation, terminal of pregnancy, fetal demise, feticide prior to labor admission, and prior cesarean delivery (e.g., trial of labor after cesarean delivery). Records that include one or more of the exclusion criteria may be excluded due to representing a different population, with unique features such as the indication for prior CD, thus requiring a distinct ML model. It is noted that in some embodiments, the ML model may be trained using a training dataset that includes records with exclusion criteria. In some embodiments the exclusion criteria and exclusion criteria described herein may be swapped, such that the training dataset includes records with the exclusion criteria, and excludes records with the inclusion criteria, for example, for training specialized ML models for specialized cases.

Alternatively or additionally, records associated with one or more of the following (optionally all of the following) are included in the training dataset: a singleton pregnancy, >=34 weeks of gestation, admitted for vaginal delivery, and fetus at vertex presentation.

For example, records of sample women carrying a singleton gestation at ≥34 weeks of gestation who were admitted for vaginal delivery with fetus at vertex presentation are included. Trials of VD of vertex singletons, at ≥34+0 gestational weeks, are included. Women with a prior cesarean and/or intrauterine fetal demise/termination of pregnancy were excluded.

At 212, a multi-record training dataset is created. A record of the training dataset includes fetal biometric parameter(s) (e.g., as described with reference to 202) optionally the adapted fetal biometric parameter(s) (e.g., as described with reference to 204) of the sample fetus obtained by the ultrasonography device, personal parameter(s) of the sample mother of the sample fetus (e.g., as described with reference to 206), and a ground truth indicating whether a birth of the sample fetus by the sample mother was a uCD (e.g., as described with reference to 208).

The multi-record training dataset may include records according to the inclusion and/or may exclude records according to the exclusion criteria (e.g., as described with reference to 210). At 214, a subset of features may be selected from the set of features that includes the fetal biometric parameter(s),r optionally the adapted fetal biometric parameter(s), and the personal parameter(s).

Feature selection may be performed using the training dataset, based on prediction importance, for example, using a feature selection approach. Features may be reduced by the selection of the subset of features, for example, from 62 features to 13 features as described in the “Examples” section with reference to an experiment performed by Inventors, or other numbers of features.

An exemplary feature selection approach is now described. A full set of features represents a baseline. Features highly correlated with the ground truth may be removed from the full set of features. For example features having a correlation value above a threshold, for example, Spearman >=0.7, or >=0.8, or >=0.9, or other values and/or other correlation values. Further feature selection may be performed, for example. The relative importance, for example in terms of relative contribution towards the ML model generating the outcome, is computed for each feature, for example, using the Random Forest machine learning approach. Features greater than a threshold (e.g., 0.5%, or 1%, or 2%, or other value) may be selected as the subset for training the ML model. Additional feature selection may be performed with the consideration of clinical knowledge, for example, by domain specific experts such as obstetricians.

In an example, the subset of selected features include at least: the number of prior VDs, cervical dilation, spontaneous onset of labor, cervical effacement and BMI at admission to labor. These features were identified as the features with the highest importance by Inventors in a study, as described with reference to the “Examples” section below. The feature spontaneous onset of labor may be replaced with the feature mechanism of onset of labor, which may include the following exemplary categories: spontaneous, prelabor rupture, and induction.

At 216, the machine learning model is trained on the multi-record training dataset, optionally using the selected subset of features. The multi-record training dataset may be created as described herein, and/or an existing multi-record training dataset may be received and/or accessed (e.g., received from another device, accessed from a data storage device, provided from another source). The trained ML model is designed for generating an outcome indicating likelihood of uCD for a target mother in response to an input of fetal biometric parameter(s) of a target fetus of the target mother and personal parameter(s) of the target mother.

The ML model may be implemented, for example, as a binary classifier that generates the outcome indicative of uCD or non-uCD (e.g., vaginal delivery). The ML model may be implemented, for example, as a multi-category classifier that generates the outcome as a category selected from multiple categories, for example, low risk of uCD, medium risk of uCD, and high risk of uCD.

The ML model may generate a predicted probability of uCD for the outcome, for example, a percentage and/or decimal value.

The ML model may be implemented as, for example, XGBoost, Distributed Random Forest (DRF), Gradient Boosting Machine (GBM), and Extremely Randomized Trees (XRT).

The ML model may be implemented using other architectures, for example, a classifier, a statistical classifier, one or more neural networks of various architectures (e.g., convolutional, fully connected, deep, encoder-decoder, recurrent, graph, combination of multiple architectures), support vector machines (SVM), logistic regression, k-nearest neighbor, decision trees, boosting, random forest, a regressor and the like.

Hyper-parameters tuning of the ML model may be performed, for example, using random search (e.g., with 5-fold cross validation). Missing values may not be imputed during the ML model training as they are interpreted as containing information (i.e., missing for a reason), rather than missing at random. Missing values may be treated as a separate category.

A validation dataset may be used for ML model evaluation and/or refinement. A test dataset may be used for final evaluation of the ML model. Validation dataset may be used to define the threshold of risk groups—subsets of the population that were classified based on their percentiles of the uCD predictions. The chosen probability thresholds of the risk groups may be implemented on the test dataset. The performance evaluation of the validation and/or the test datasets may be performed per each risk group.

The ML model may be evaluate for clinical discrimination across gestational ages and/or major clinical risk factors for uCD, by evaluated the ML model's PPV and NPV in the following subgroups: GA at admission to labor ≥34-<37 gestational weeks, >37 gestational weeks, advanced maternal age (e.g., defined as ≥40 years), obesity (e.g., defined as BMI at admission to labor ≥30 kg/m 2), nulliparity, and/or the need for induction of labor.

At 218, the ML model may be analyzed.

The ML model may be analyzed, for example, to identify the features that most significantly contribute to the outcome of the ML model. A machine learning model interpretability process may be applied to the ML model for analyzing the personal parameters and/or the fetal biometric parameters (e.g., the selected subset of parameter) for identifying the subset of the personal parameters and/or the subset of the fetal biometric parameters indicating most significant contribution to the outcome generated by the machine learning model. For example, features having a relative contribution and/or absolute contribution above a threshold. The identified subset of the personal parameters and/or the subset of the fetal biometric parameters that most significantly contribute to the outcome may be further selected as the features used to train (or re-train) the ML model, as described with reference to 214. Alternatively or additionally, the identified subset of the personal parameters and/or the subset of the fetal biometric parameters that most significantly contribute to the outcome may be identified as a critical subset of features that are required as input into the trained ML model. The ML model may not generate the outcome if any of these critical features are missing.

In an example, a SHAP (SHapley Additive exPlanations) based approach may be used as the machine learning model interpretability process. A SHAP summary plot for the ML model (e.g., XGBoost model) may be created. SHAP quantifies the contribution of each feature to the ML model prediction. The SHAP plot combines features' importance with features' effect.

Exemplary features identified as most significant contributors to the outcome of the ML model (e.g., as discovered by Inventors in the study described in the “Examples” section below): Number of prior vaginal deliveries, Cervical dilation, Spontaneous onset of labor and Maternal height are negatively correlated with uCD occurrence, while the features: Maternal age, Maternal BMI at admission to labor and GA at admission are positively correlated with uCD occurrence. Inventors observed, that the number of prior VDs, cervical dilation, spontaneous onset of labor and maternal height were predictive of VD success. On the other hand, maternal age, maternal BMI and GA were predictive of uCD.

Referring now back to FIG. 3, the ML model may be used to obtain outcomes for target individuals, for example, women carrying a singleton pregnancy with the exception of women attempting TOLAC (Trial Of Labor After Cesarean delivery). The ML model may be used for target individuals carrying features of a wide range of gestations ages and/or a wide range of risk groups including nulliparous women, advanced maternal age, women suffering from obesity, and/or women requiring induction of labor.

at 302, fetal biometric parameter(s) of a target fetus of a target mother obtained by an ultrasonography device, are accessed. Details of fetal biometric parameter(s), examples thereof, and/or exemplary approaches for obtaining the fetal biometric parameter(s) are described herein, for example, with reference to 202 of FIG. 2.

The fetal biometric parameter(s) may be obtained at time of admission of the sample mother to labor and/or other event indicating impending birth.

At 304, the fetal biometric parameter(s) are adapted to create adapted fetal biometric parameter(s).

The fetal biometric parameter(s) may be adapted when the fetal biometric parameter(s) of the target fetus obtained by the ultrasonography device is of a historical gestational age of the target fetus. The adaptation is performed to compute an adapted fetal biometric parameter(s) depicting a current gestational age at time of admission to labor.

Exemplary details of adapting the fetal biometric parameter(s) are described herein, for example, with reference to 204 of FIG. 2.

At 306, personal parameter(s) of the target mother are accessed. Details of personal parameter(s), examples thereof, and/or exemplary approaches for obtaining the personal parameter(s) are described herein, for example, with reference to 206 of FIG. 2.

The personal parameter(s) may be obtained at time of admission of the sample mother to labor and/or other event indicating impending birth.

At 308, the fetal biometric parameter(s), optionally the adapted fetal biometric parameter(s) and the personal parameter(s) are fed into the ML model (e.g., trained as described with reference to FIG. 2).

Optionally, the parameter(s) fed into the ML model are selected according to the selected subset of features (e.g., described with reference to 214 of FIG. 2) and/or according to the features identified as most significantly contributing to the outcome of the ML model (e.g., described with reference to 218 of FIG. 2).

Features of target subjects which satisfy the inclusion criteria (e.g., described with reference to 210 of FIG. 2) may be fed into the ML model. Features of target subjects which satisfy the exclusion criteria (e.g., described with reference to 210 of FIG. 2) may be rejected, and/or the specialized ML model trained for such specific cases may be used.

At 310, an outcome indicating likelihood of uCD for the target mother is obtained from the machine learning model. The outcome may be a binary category, for example, uCD or non-uCD (e.g., vaginal delivery). The outcome may be a category from multiple categories, for example, low likelihood of uCD, medium likelihood of uCD, and high likelihood of uCD. The outcome may be and/or may include a probability of uCD, for example, a percentage and/or other numerical value (e.g., 73%, 0.73, and the like).

At 312, the ML model may be analyzed, for computing relative contribution by the fetal biometric parameter(s) and/or by the personal parameter(s) towards the generated outcome. The ML model may be analyzed by applying a machine learning interpretability process, for example, a SHAP based approach, as described with reference to 218. Optionally, a relative contribution and/or positive or negative correlation (e.g., positive or negative SHAP value) towards the outcome is computed for the inputted parameters, optionally for each inputted parameters.

At 314, the outcome is provided, for example, the outcome is presented on a display of a client terminal, the outcome is fed into another executed process (e.g., automatically ordering tests), the outcome is stored on a data storage device (e.g., in a field of a health record of the target individual), and/or the outcome is forwarded to another device (e.g., to an administrative server viewed by the on-call physician).

The outcome may be provided for presentation on a dashboard presented on a display. The dashboard may be presented within a graphical user interface that acts as a monitoring and/or control station for monitoring patients. For example, the dashboard may be presented on a display of a computer at the nurse's station and/or on a mobile device of the attending physician and/or on a display of a server used by the healthcare staff.

Optionally, the relative contribution by the inputted parameters (e.g., each parameter) is provided, for example, presented on the display of the client terminal. The relative contribution presented on the display may be used, for example, to help the mother and/or physician understand the most significant features that led the ML model for generating the outcome. The relative contribution of the most significant features may be to help explain to the mother, for example, why cesarean delivery is recommended (e.g., presence of significant feature that led the ML model to generate an outcome indicating high risk of uCD). In another example, to reassure the mother that a birth at home is safe, for example, no features that are significant for uCD exist.

At 316, one or more actions may be triggered in response to the outcome.

The actions may be automatic, for example, in response to an outcome indicating likelihood of uCD, a message is sent to an on-call surgeon for a consultation and/or to an anesthesiologist for a pre-operation evaluation. In another example, in response to an outcome indicating low likelihood of uCD, a labor delivery room may be automatically scheduled for the subject. In another example, an automated video, message, and/or animation (e.g., interactive) may be presented on a client terminal, to aid the target mother in making a decision. For example, when the risk of uCD is low, a video reassuring the mother of the low risk may be played. When the risk of uCD is high, an interactive GUI helping the mother make a decision of whether to proceed with vaginal delivery or go directly to cesarean delivery may be presented on a mobile device of the mother.

The actions may be manual, for example, the physician and/or nurse may provide guidance and/or recommendations to the subject. For example, women with high likelihood of uCD may be advised to undergo a planned cesarean delivery rather than risk uCD. Women with low likelihood of uCD suffering from anxiety may be reassured that the risk of uCD is low.

For example, as described in the study in the “Examples” section, approximately fifty percent of deliveries were stratified to the lowest risk group that were at risk of 1% or less for uCD among the test group. Such results may trigger, for example, an automatic message of reassurance and/or a manual reassurance consultation session by a nurse. Approximately one percent of deliveries were stratified to the highest risk group that are at 58% risk of uCD. Such results may trigger, for example, an automatic request for a consultation with an obstetrical surgeon and/or anesthesiologist. In between, women at risk percentiles of 51-99% were at increasing risk for uCD ranging between 5-32% that is calculated for each group separately. Such results may trigger, for example, an interactive GUI to help the woman decide how to proceed, and/or a consultation with a psychiatrist and/or member of the obstetrical care team to help with the decision making process.

It is noted that a high probability for uCD does not mandate elective CD, but rather serves for counseling women at time of admission to labor.

The ML model allows for women's individualized decision making (28), for example, weighing individual probabilities with individual preferences and/or individual probability of complications in the case uCD indeed occurs. For instance, the incision to delivery time, neonatal and maternal morbidity are increased in women suffering from obesity compared to normal weight women (29, 30).

At 318, the subject, i.e., the target mother, is treated according to the outcome generated by the ML model. Optionally, in response to obtaining the outcome of the machine learning model indicating high likelihood of uCD, the target mother is treated by performing a cesarean delivery, i.e., a surgical procedure to deliver the baby through incisions in the abdomen and uterus. The high likelihood of uCD may be, for example, a value of the outcome (e.g., probability of uCD) above a threshold indicating high likelihood (e.g., greater than 70%, or 80%, or 90%, or other values), and/or a classification category outcome of the uCD indicating high likelihood.

Alternatively, in response to obtaining the outcome of the machine learning model indicating low likelihood of uCD, the target mother is treated by performing a vaginal delivery, i.e., non-Cesarean Delivery. The vaginal delivery may be performed at home when the outcome indicates low likelihood of uCD.

Various embodiments and aspects as delineated hereinabove and as claimed in the claims section below find experimental and/or calculated support in the following examples.

### Examples

Reference is now made to the following examples, which together with the above descriptions illustrate some embodiments of the invention in a non limiting fashion.

Inventors developed and trained an ML model using real patient data. The ML model was evaluated and/or validated using additional real patient data, as described below.

### Materials and Methods

### Patients

The study cohort consisted of deliveries that occurred at The Sheba Medical Center, a university affiliated tertiary medical center, between March 2011 and June 2020. Inventors reviewed the electronic health records (EHR) of all women who delivered during the study period to identify all deliveries that met the inclusion criteria. These included trials of vaginal delivery (VD) of vertex singletons, at ≥34+0 gestational weeks. Exclusion criteria were intrauterine fetal demise or feticide prior to labor admission as well as of trial of labor after cesarean delivery (CD). Records meeting the exclusion criteria were excluded because they comprise a different population, with unique features such as the indication for prior CD, thus requiring a distinct ML model.

### Data Collection

Inventors collected baseline maternal characteristics including age, prepregnancy weight, height, body mass index (BMI), smoking status and maternal comorbidities; obstetrical history; current pregnancy characteristics including gestational age (GA) at admission to labor, pregnancy complications (gestational diabetes mellitus, hypertensive disorders of pregnancy, intrahepatic cholestasis of pregnancy); fetal features including sex and ultrasound parameters. These included four fetal sonographic biometric measurements including biparietal diameter (BPD), head circumference (HC), abdominal circumference (AC) and femur length (FL), the estimated fetal weight (EFW) derived from these measurements, and amniotic fluid index (AFI); and features available at admission to labor including weight, cervical dilation and effacement, fetal station and mode of start of delivery.

The BMI was calculated as weight (kg)/height² (m). Weight was reported by the parturient as pre-gestational and weight at admission to labor. Hypertensive disorders were defined according to the American College of Obstetricians and Gynecologists (ACOG) (23). Diabetic disorders were defined as either pregestational diabetes, in accordance with the American Diabetes Association criteria (24), or gestational diabetes mellitus, using the diagnostic thresholds established by Carpenter and Coustan (25). AFI was calculated by summing the maximal vertical amniotic fluid pocket diameter in four quadrants of the uterus. In cases that cervical ripening was required, either intracervical foley catheter or prostaglandin E2 were used at the discretion of the treating physician.

### Adjusted Sonographic Biometry According to Gestational Age

As fetal ultrasound biometry may change from the examination and the delivery, and due to the effect of fetal biometrical parameters on VD success rate, Inventors adjusted the biometric parameters according to the GA at admission to labor. Inventors have previously described the sonographic biometry adjustment in detail (20). In brief, the fetal ultrasound parameters' adjustment was performed using the following steps. First, Inventors calculated the percentile of EFW at the GA it was performed, based on the Hadlock equations (26, 27), by calculating the median expected EFW for the GA on the day the ultrasound was performed. For every observation, Inventors calculated the EFW at admission to labor based on the normal distribution, the percentile which was calculated in the previous step, and the median expected EFW for the GA at the day of delivery. These steps were used to adjust EFW to aEFW. The same steps were followed to adjust BPD, HC, AC and FL to aBPD, aHC, aAC and aFL, respectively.

### Machine Learning Model and Statistical Analysis

Clinical characteristics were reported within the group of women who achieved VD and women who underwent uCD. The clinical characteristics were expressed as percentages for categorical features and as median and interquartile range (IQR) for continuous features. Kruskal-Wallis test was performed for continuous features and Chi-squared test for categorical features (Fisher's exact test in case of small numbers). Logistic regression was used for calculating unadjusted odds ratio (OR), with 95% confidence interval (CI) for uCD.

The population for the model generation was divided into training, validation and test datasets. Deliveries between March 2011 and May 2019 were allocated to the training (80%) and validation datasets (20%) in a random fashion. Deliveries between June 2019 and June 2020 were allocated to the test dataset.

The feature selection was performed using the training dataset. Inventors started with a panel of 62 features. Highly correlated (Spearman>=0.8) features were removed from the model's panel of features. Further feature selection was performed using Random Forest machine learning. Features with relative importance of more than 1% were selected to be part of the ML model. At last, additional feature selection was performed with the consideration of clinical knowledge.

The ML model building was performed using the training dataset. Four ML models, XGBoost, Distributed Random Forest (DRF), Gradient Boosting Machine (GBM), Extremely Randomized Trees (XRT) were examined to predict uCD using the selected features.

Referenced is now made to FIG. 4, which is graph representing performance of the four ML models trained and evaluated as part of the experiment, XGBoost, GBM, XRT, and DRF, in accordance with some embodiments of the present invention. The graph depicted in FIG. 4 indicates that XGBoost model performed best based on the receiver operator characteristic (ROC) area under the curve (AUC) results. CI denotes confidence interval. Hyper-parameters tuning of the XGBoost model was performed using random search (with 5-fold cross validation). For the current study, the maximum depth of each tree was set to 5 and the number of trees was 1,000. Missing values were not imputed during the ML model training as they were interpreted as containing information (i.e., missing for a reason), rather than missing at random. Missing values were treated as a separate category.

The validation dataset was used for ML model evaluation and refinement and the test dataset was used for final evaluation of the ML model. Validation dataset was also used to define the threshold of risk groups—subsets of the population that were classified based on their percentiles of the uCD predictions. The chosen probability thresholds of the risk groups were implemented on the test dataset. The performance evaluation of the validation and the test datasets were performed per each risk group.

The ROC AUC served as the primary metric of the model accuracy while positive predictive value (PPV) and negative predictive value (NPV) served as the primary metric of the model's clinical discrimination

To evaluate the ML model's clinical discrimination across gestational ages and major clinical risk factors for uCD, Inventors evaluated the model's PPV and NPV in the following subgroups: GA at admission to labor ≥34-<37 gestational weeks, >37 gestational weeks, advanced maternal age (defined as ≥40 years), obesity (defined as BMI at admission to labor ≥30 kg/m 2), nulliparity and the need for induction of labor.

Statistical analyses were performed using Python version 3.6.7. Machine learning models were built using H2O package version 3.22

### Ethical Approval

The study protocol was approved by the Sheba Medical Center Committee for Human Subjects Research (#7145-20-SMC, 30 Sep. 2020).

### Results

### Demographics

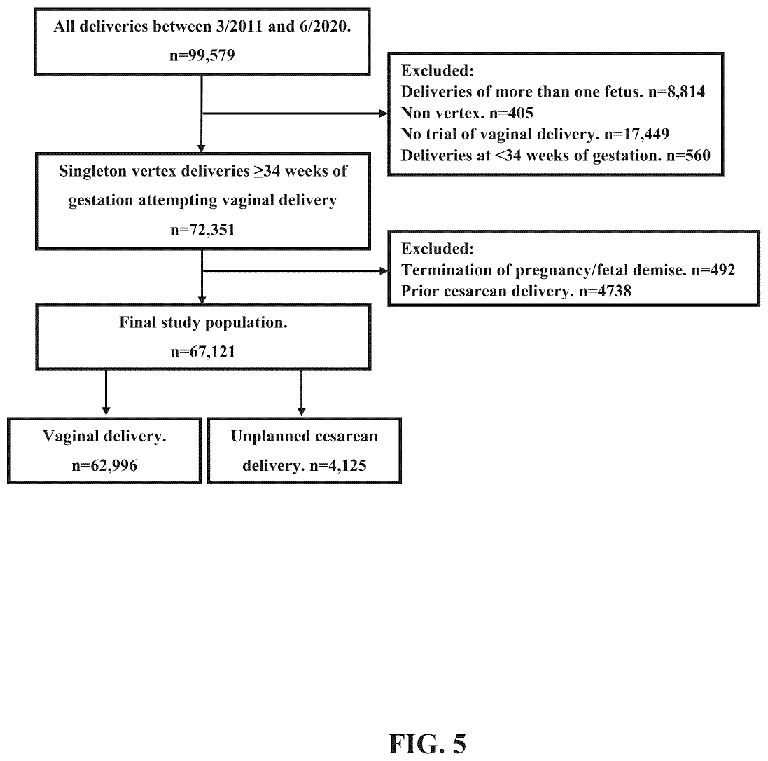

Reference is now made to FIG. 5, which is a flowchart depicting exclusion and inclusion criteria of the study cohort, in accordance with some embodiments of the present invention. During the study period 99,579 deliveries took place at the medical center. Based on the described inclusion and exclusion criteria, 67,121 deliveries remained in the final study cohort.

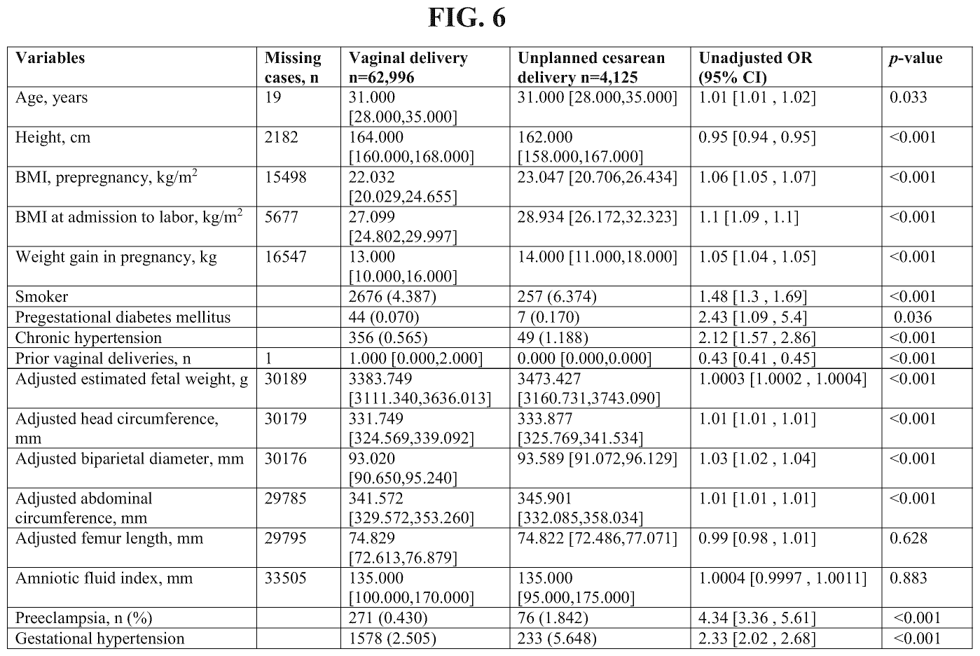

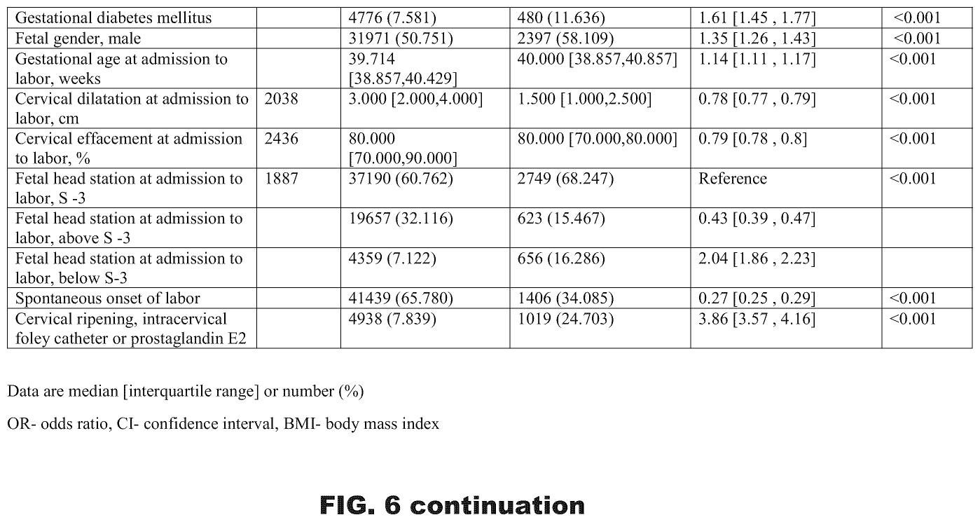

Reference is now made to FIG. 6, which is a table presenting Characteristics of the study groups: vaginal delivery versus unplanned cesarean delivery, of the study, in accordance with some embodiments of the present invention. Overall, 62,996 (93.9%) of the TOL attempts were delivered vaginally, and 4,125 (6.1%) VD attempts resulted in uCDs. Women in the VD group were younger, taller, and with a lower weight and BMI compared with women who underwent uCD. The median number of prior VDs was higher in the VD group. Diabetic and hypertensive disorders rates were lower in the VD group. The aEFW, aHC, aBPD and aAC were lower in the VD group, while aFL and amniotic fluid index did not differ between groups. GA at admission to labor was lower in the VD group. Cervical dilation and effacement were higher in the VD group. Fetal station at admission to labor was lower in the pelvis in the VD group. The rate of spontaneous onset of labor was higher in the VD group.

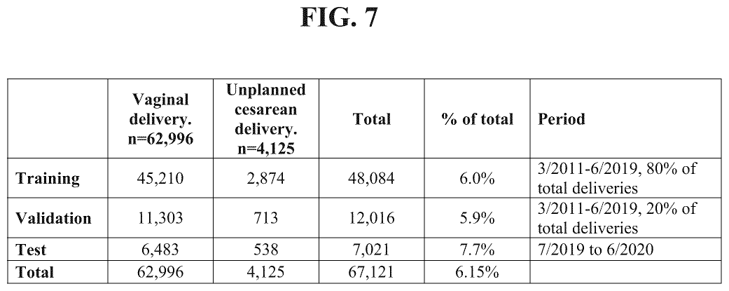

Reference is now made to FIG. 7, which is a table presenting a training-validation-test split of the data of the study, in accordance with some embodiments of the present invention. The training, validation and test sets comprised 48,084, 12,016 and 7,021 cases, respectively. The proportion of uCDs were 6.0%, 5.9% and 7.7% among each of the groups, respectively.

### Unplanned Cesarean Delivery Prediction Model

### Features Selection

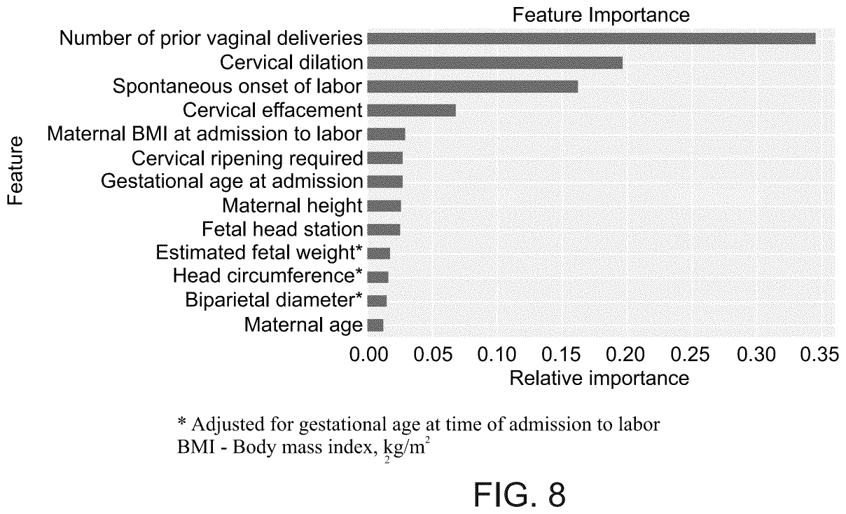

Reference is now made to FIG. 8, which is a graph presenting selected features for the ML model of the study, in accordance with some embodiments of the present invention. Thirteen features were selected for the final ML model, presented in their order of importance. The number of prior VDs, cervical dilation, spontaneous onset of labor, cervical effacement and BMI at admission to labor were identified as the features with the highest importance.

### ML Model Explainability

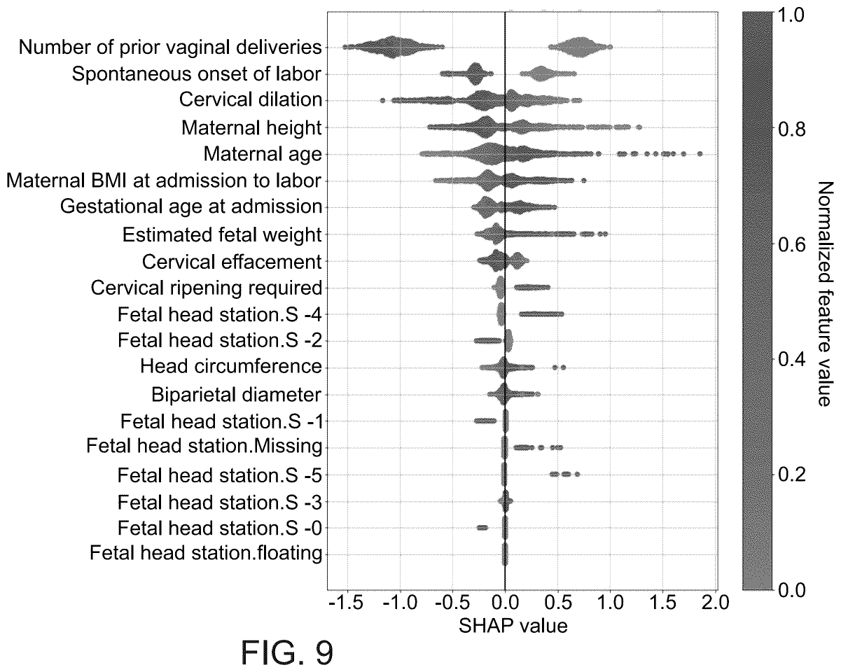

Reference is now made to FIG. 9, which is a SHAP (SHapley Additive exPlanations) summary plot for the XGBoost model of the study, in accordance with some embodiments of the present invention. SHAP quantifies the contribution of each feature to the model prediction. The SHAP plot combines features' importance with features' effect. The features are ordered according to their importance. For example, ‘Number of prior vaginal deliveries’ has a high importance and a negative correlation with uCD. The “high” is represented by the red color, and the “negative” impact is represented by the colors' location on the X-axis. The features: Number of prior vaginal deliveries, Cervical dilation, Spontaneous onset of labor and Maternal height were all negatively correlated with uCD occurrence, while the features: Maternal age, Maternal BMI at admission to labor and GA at admission are positively correlated with uCD occurrence.

### Validation Dataset

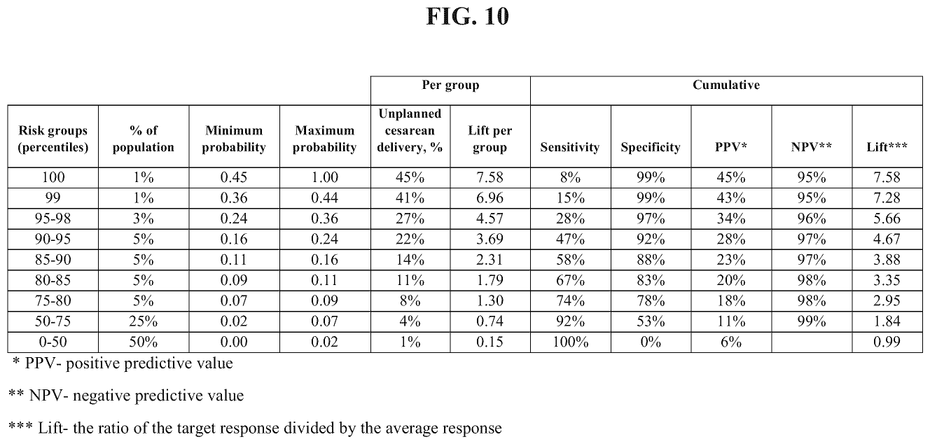

Reference is now made to FIG. 10, which is a table of the performance of the ML model for the validation dataset (n=12,016) of the study, in accordance with some embodiments of the present invention.

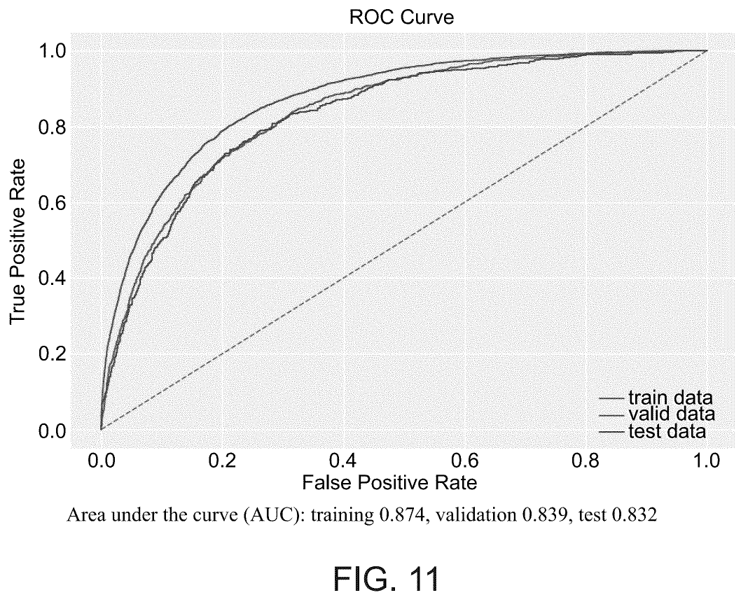

Reference is now made to FIG. 11, which is a receiver operating characteristic (ROC) curve for the prediction of vaginal delivery of the ML model of the study, in accordance with some embodiments of the present invention. The area under the ROC curve (AUC) was 0.839. Additionally, inventors observed the model's potential clinical discrimination. Women at the 100^{th} risk percentile group were at 45% risk for uCD. In contrast, half the population, ≤50^{th} percentile risk group, had a NPV of ≥99% for uCD, i.e. ≤1% risk for uCD.

### Test Dataset

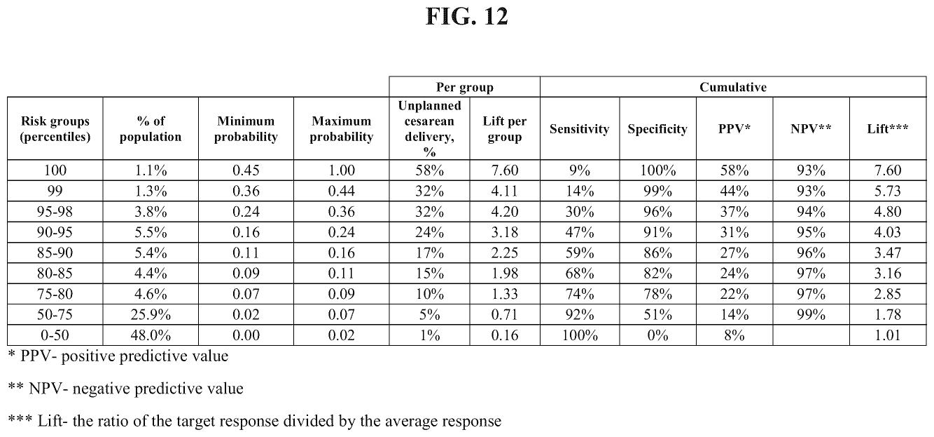

Reference is now made to FIG. 12, which is a table summarizing the performance of ML model for the test dataset (n=7021) of the study, in accordance with some embodiments of the present invention.

Referring back to FIG. H, the AUC among the test dataset was 0.832. In the test dataset, women at the 100^{th} risk percentile group were at 58% risk for uCD. Similarly to the validation dataset, the ≤50^{th} percentile risk group had a negative predictive value of ≥99% for uCD.

### Sub-Group Analyses

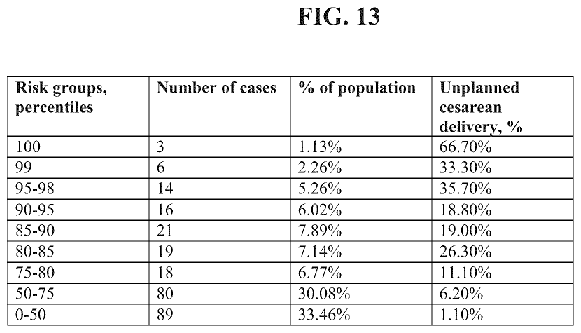

Reference is now made to FIG. 13, which is a table summarizing performance of the ML model for test cohorts that includes deliveries ≥34 and <37 weeks of gestation of the study, in accordance with some embodiments of the present invention. This sub-group of the test cohort consisted of 266 cases. Women at the 100^{th} risk percentile group were at 66.7% risk for uCD. The ≤50^{th} percentile risk group had a risk of 1.10% for uCD.

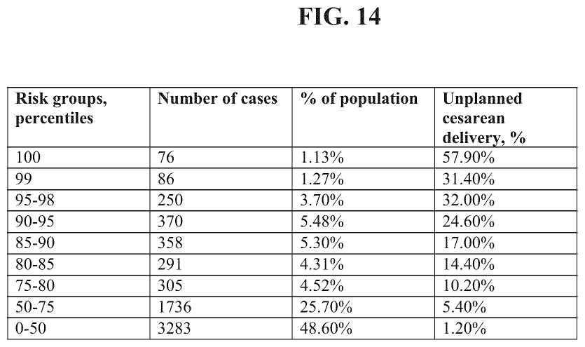

Reference is now made to FIG. 14, which is a table summarizing performance of the ML model for the test cohort that includes deliveries ≥37 weeks of gestation of the study, in accordance with some embodiments of the present invention. This sub-group of the test cohort consisted of 6,755 deliveries. Women at the 100^{th} risk percentile group were at 57.9% risk for uCD. The ≤50^{th} percentile risk group had a risk of 1.20% for uCD.

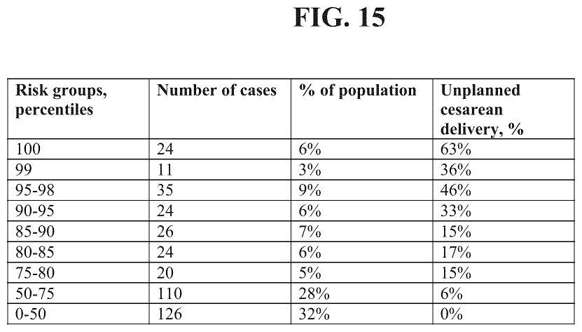

Reference is now made to FIG. 15, which is a table summarizing performance of the ML model for the test cohort that includes women of the study that delivered at an advanced maternal age, in accordance with some embodiments of the present invention. This subgroup consisted of 400 deliveries. Women at the 100^{th} risk percentile group were at 63% risk for uCD. There were no uCD among the ≤50^{th} percentile risk group.

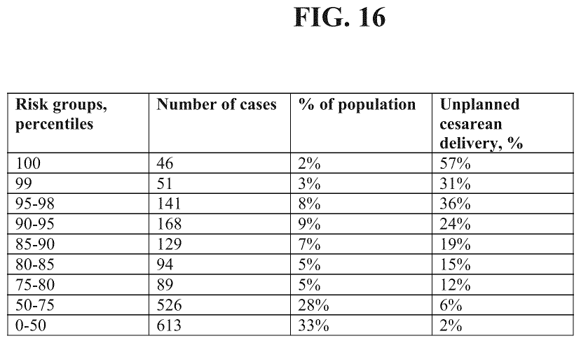

Reference is now made to FIG. 16, which is a table summarizing performance of the ML model for the test cohort that includes obese women, in accordance with some embodiments of the present invention. This subgroup consisted of 1,857 deliveries. Women at the 100^{th} risk percentile group were at 57% risk for uCD. The ≤50^{th} percentile risk group had a risk of 2% for uCD.

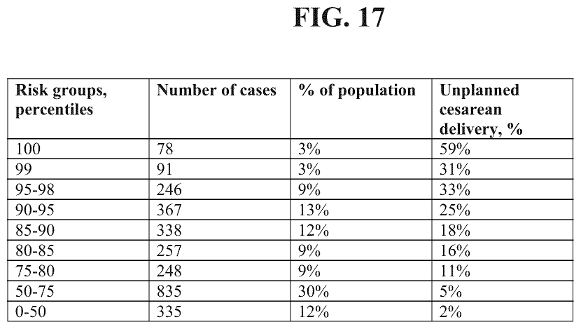

Reference is now made to FIG. 17, which is a table summarizing performance of the ML model for the test cohort that includes nulliparous women, in accordance with some embodiments of the present invention. This subgroup consisted of 2,795 deliveries. Women at the 100^{th} risk percentile group were at 59% risk for uCD. The ≤50^{th} percentile risk group had a risk of 2% for uCD.

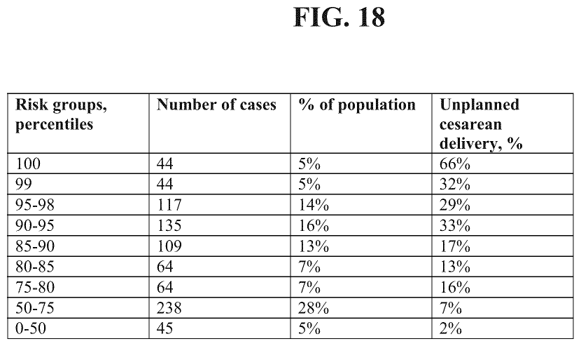

Reference is now made to FIG. 18, which is a table summarizing performance of the ML model for the test cohort that includes women undergoing induction of label, in accordance with some embodiments of the present invention. This sub-group consisted of 860 deliveries. Women at the 100^{th} risk percentile group were at 66% risk for uCD. The ≤50^{th} percentile risk group had a risk of 2% for uCD.

### REFERENCES

1. Rosenbloom J I, Stout M J, Tuuli M G, Woolfolk C L, López J D, Macones G A, et al. New labor management guidelines and changes in cesarean delivery patterns. Am J Obstet Gynecol. 2017; 217(6):689.e1-.e8.
2. Haile Z T, Chavan B, Teweldeberhan A K, Chertok I R A, Francescon J. Gestational weight gain and unplanned or emergency cesarean delivery in the United States. Women Birth. 2019; 32(3):263-9.
3. Burke N, Burke G, Breathnach F, McAuliffe F, Morrison J J, Turner M, et al. Prediction of cesarean delivery in the term nulliparous woman: results from the prospective, multicenter Genesis study. Am J Obstet Gynecol. 2017; 216(6):598.e1-.e11.
4. Rose A, Raja E A, Bhattacharya S, Black M. Intervention thresholds and cesarean section rates: A time-trends analysis. Acta Obstet Gynecol Scand. 2018; 97(10):1257-66.
5. Levine L D, Downes K L, Parry S, Elovitz M A, Sammel M D, Srinivas S K. A validated calculator to estimate risk of cesarean after an induction of labor with an unfavorable cervix. Am J Obstet Gynecol. 2018; 218(2):254.e1-.e7.
6. Wilson-Leedy J G, DiSilvestro A J, Repke J T, Pauli J M. Reduction in the Cesarean Delivery Rate After Obstetric Care Consensus Guideline Implementation. Obstet Gynecol. 2016; 128(1):145-52.
7. Alexander J M, Leveno K J, Rouse D J, Landon M B, Gilbert S, Spong C Y, et al. Comparison of maternal and infant outcomes from primary cesarean delivery during the second compared with first stage of labor. Obstet Gynecol. 2007; 109(4):917-21.
8. Allen V M, O'Connell C M, Liston R M, Baskett T F. Maternal morbidity associated with cesarean delivery without labor compared with spontaneous onset of labor at term. Obstet Gynecol. 2003; 102(3):477-82.
9. Allen V M, O'Connell C M, Baskett T F. Maternal and perinatal morbidity of caesarean delivery at full cervical dilatation compared with caesarean delivery in the first stage of labour. BJOG. 2005; 112(7):986-90.
10. Vitner D, Bleicher I, Levy E, Sloma R, Kadour-Peero E, Bart Y, et al. Differences in outcomes between cesarean section in the second versus the first stages of labor. J Matern Fetal Neonatal Med. 2019; 32(15):2539-42.
11. Pergialiotis V, Vlachos D G, Rodolakis A, Haidopoulos D, Thomakos N, Vlachos G D. First versus second stage C/S maternal and neonatal morbidity: a systematic review and meta-analysis. Eur J Obstet Gynecol Reprod Biol. 2014; 175:15-24.
12. Selo-Ojeme D, Sathiyathasan S, Fayyaz M. Caesarean delivery at full cervical dilatation versus caesarean delivery in the first stage of labour: comparison of maternal and perinatal morbidity. Arch Gynecol Obstet. 2008; 278(3):245-9.
13. Rossi R M, Requarth E, Warshak C R, Dufendach K R, Hall E S, DeFranco E A. Risk Calculator to Predict Cesarean Delivery Among Women Undergoing Induction of Labor. Obstet Gynecol. 2020; 135(3):559-68.
14. Rossi R M, Requarth E W, Warshak C R, Dufendach K, Hall E S, DeFranco E A. Predictive Model for Failed Induction of Labor Among Obese Women. Obstet Gynecol. 2019; 134(3):485-93.
15. Eggebo T M, Wilhelm-Benartzi C, Hassan W A, Usman S, Salvesen K A, Lees C C. A model to predict vaginal delivery in nulliparous women based on maternal characteristics and intrapartum ultrasound. Am J Obstet Gynecol. 2015; 213(3):362.e1-6.
16. Fergus P, Hussain A, Al-Jumeily D, Huang D S, Bouguila N. Classification of caesarean section and normal vaginal deliveries using foetal heart rate signals and advanced machine learning algorithms. Biomed Eng Online. 2017; 16(1):89.
17. Fergus P, Selvaraj M, Chalmers C. Machine learning ensemble modelling to classify caesarean section and vaginal delivery types using Cardiotocography traces. Comput Biol Med. 2018; 93:7-16.
18. Bzdok D, Altman N, Krzywinski M. Statistics versus machine learning. Nat Methods. 2018; 15(4):233-4.
19. Beam A L, Kohane I S. Big Data and Machine Learning in Health Care. JAMA. 2018; 319(13):1317-8.
20. Tsur A, Batsry L, Toussia-Cohen S, Rosenstein M G, Barak O, Brezinov Y, et al. Development and validation of a machine-learning model for prediction of shoulder dystocia. Ultrasound Obstet Gynecol. 2020; 56(4):588-96.
21. Meyer R, Hendin N, Zamir M, Mor N, Levin G, Sivan E, et al. Implementation of machine learning models for the prediction of vaginal birth after cesarean delivery. J Matern Fetal Neonatal Med. 2020:1-7.
22. Guedalia J, Lipschuetz M, Novoselsky-Persky M, Cohen S M, Rottenstreich A, Levin G, et al. Real-time data analysis using a machine learning model significantly improves prediction of successful vaginal deliveries. Am J Obstet Gynecol. 2020; 223(3):437.e1-.e15.
23. ACOG Practice Bulletin No. 202: Gestational Hypertension and Preeclampsia. Obstet Gynecol. 2019; 133(1):e1-e25.
24. Association A D. Standards of medical care in diabetes--2011. Diabetes Care. 2011; 34 Suppl 1:S11-61.
25. Carpenter M W, Coustan D R. Criteria for screening tests for gestational diabetes. Am J Obstet Gynecol. 1982; 144(7):768-73.
26. Hadlock F P, Harrist R B, Carpenter R J, Deter R L, Park S K. Sonographic estimation of fetal weight. The value of femur length in addition to head and abdomen measurements. Radiology. 1984; 150(2):535-40.
27. Hadlock F P, Harrist R B, Martinez-Poyer J. In utero analysis of fetal growth: a sonographic weight standard. Radiology. 1991; 181(1):129-33.
28. Bukowski R, Schulz K, Gaither K, Stephens K K, Semeraro D, Drake J, et al. Computational medicine, present and the future: obstetrics and gynecology perspective. Am J Obstet Gynecol. 2021; 224(1):16-34.
29. Conner S N, Tuuli M G, Longman R E, Odibo A O, Macones G A, Cahill A G. Impact of obesity on incision-to-delivery interval and neonatal outcomes at cesarean delivery. Am J Obstet Gynecol. 2013; 209(4):386.e1-6.
30. Lauth C, Huet J, Dolley P, Thibon P, Dreyfus M. Maternal obesity in prolonged pregnancy: Labor, mode of delivery, maternal and fetal outcomes. J Gynecol Obstet Hum Reprod. 2021; 50(1): 101909.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

It is expected that during the life of a patent maturing from this application many relevant machine learning models will be developed and the scope of the term machine learning model is intended to include all such new technologies a priori.

As used herein the term “about” refers to ±10%.

The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A computer implemented method of training a machine learning model for prediction of unplanned Cesarean Delivery (uCD), comprising:
creating or receiving a multi-record training dataset, wherein a record comprises:
at least one fetal biometric parameter of a sample fetus obtained by an ultrasonography device,
at least one personal parameter of a sample mother of the sample fetus, and
a ground truth indicating whether a birth of the sample fetus by the sample mother was an uCD during attempted vaginal delivery or via vaginal delivery; and
training the machine learning model on the multi-record training dataset for generating an outcome indicating likelihood of uCD for a target mother in response to an input of at least one fetal biometric parameter of a target fetus of the target mother and at least one personal parameter of the target mother.

2. The computer implemented method of claim 1, wherein the at least one fetal biometric parameter and the at least one personal parameter are for the fetus and/or sample mother at time of admission of the mother to labor.

3. The computer implemented method of claim 1, wherein the at least one fetal biometric parameter of the sample fetus obtained by the ultrasonography device depicts a historical gestational age of the sample fetus, and further comprising adapting the at least one fetal biometric parameter to an adapted at least one fetal biometric parameter depicting a current gestational age at time of admission to labor, wherein the record includes the adapted at least one fetal biometric parameter.

4. The computer implemented method of claim 1, wherein the at least one personal parameter comprises at least one risk modifier.

5. The computer implemented method of claim 1, wherein the at least one personal parameter is based on a state of a cervix of the mother.

6. The computer implemented method of claim 1, wherein the at least one personal parameter is represented as a continuous value.

7. The computer implemented method of claim 1, wherein the at least one fetal biometric parameter is selected from a group comprising: estimated fetal weight, head circumference, and biparietal diameter.

8. The computer implemented method of claim 1, wherein the at least one personal parameter is selected from a group comprising: number of prior vaginal deliveries, cervical dilation, spontaneous onset of labor, cervical effacement, maternal BMI at admission to labor, cervical ripening required, gestational age at admission, maternal height, fetal head station, and maternal age.

9. (canceled)

10. The computer implemented method of claim 1, wherein the machine learning model comprises a binary classifier that generates the outcome indicative of uCD or vaginal delivery.

11. The computer implemented method of claim 1, wherein the outcome indicating likelihood of uCD generated by the machine learning model comprises a predicted probability of uCD.

12. The computer implemented method of claim 1, further comprising excluding records from the training dataset associated with personal parameters that include values of at least one of: a delivery of more than one fetus, non-vertex, no trial of vaginal delivery, delivery at <34 weeks of gestation, terminal of pregnancy, fetal demise, and prior cesarean delivery.

13. The computer implemented method of claim 1, further comprising including records in the training dataset with personal parameters including values of: a singleton pregnancy, >=34 weeks of gestation, admitted for vaginal delivery, and fetus at vertex presentation.

14. The computer implemented method of claim 1, further comprising computing relative importance of the at least one fetal biometric parameter and/or for the at least one personal parameter in generating the outcome by the ML model, selecting a subset of the at least one fetal biometric parameter and/or for the at least one personal parameter having a relative importance above a threshold, wherein records of the selected subset are included in the multi-record training dataset used to train the ML model.

15. A computer implemented method of prediction of uCD, comprising:
feeding at least one fetal biometric parameter of a target fetus of a target mother obtained by an ultrasonography device and at least one personal parameter of the target mother into a machine learning model trained according to claim 1, and
obtaining an indicating likelihood of uCD for a target mother as an outcome of the machine learning model.

16. The computer implemented method of claim 15, further comprising, in response to obtaining the outcome of the machine learning model indicating high likelihood of uCD, treating the target mother by performing a cesarean delivery surgical procedure.

17. The computer implemented method of claim 15, further comprising, in response to obtaining the outcome of the machine learning model indicating low likelihood of uCD, treating the target mother by performing a vaginal delivery.

18. The computer implemented method of claim 15, further comprising: when the at least one fetal biometric parameter of the target fetus obtained by the ultrasonography device is of a historical gestational age of the target fetus, adapting the at least one fetal biometric parameter to an adapted at least one fetal biometric parameter depicting a current gestational age at time of admission to labor, wherein feeding comprises feeding the adapted at least one fetal biometric parameter into the machine learning model.

19. The computer implemented method of claim 15, wherein the at least one fetal biometric parameter and the at least one personal parameter are for the fetus and/or sample mother at time of admission of the mother to labor.

20. The computer implemented method of claim 15, further comprising applying a machine learning model interpretability process for computing relative contribution of each one of the at least one fetal biometric parameter and the at least one personal parameter towards the outcome generated by the machine learning model, and presenting an indication of the relative contribution on a display.

21. A system for prediction of uCD, comprising:
at least one processor executing a code for:
feeding at least one fetal biometric parameter of a target fetus of a target mother obtained by an ultrasonography device and at least one personal parameter of the target mother into a machine learning model, and
obtaining an indicating likelihood of uCD for a target mother as an outcome of the machine learning model,
wherein the machine learning model is trained on a multi-record training dataset, wherein a record comprises:
at least one fetal biometric parameter of a sample fetus of a sample mother obtained by an ultrasonography device,
at least one personal parameter of the sample mother, and
a ground truth indicating whether a birth of the sample fetus by the sample mother was an uCD during attempted vaginal delivery or via vaginal delivery.
